(19) United States
(12) Patent Application Publication
(10) Pub. No.: US 2025/0276296 A1
(43) Pub. Date: Sep. 4, 2025

(54) FOOD WASTE DISPOSER

(71) Applicant: Resaeng Inc., Busan (KR)

(72) Inventors: Jin Hwi BANG, Gimhae-si (KR); Jung Ho HWANG, Gimhae-si (KR); Jae Hong KIM, Busan (KR)

(73) Assignee: Resaeng Inc., Busan (KR)

(21) Appl. No.: 19/060,848

(22) Filed: Feb. 24, 2025

(30) Foreign Application Priority Data
Feb. 29, 2024 (KR) 10-2024-0029493

(51) Int. Cl. B01F 27/07; B01F 27/072; B01F 27/112; B01F 27/118; B01F 27/191; B01F 27/70; B01F 35/21; B01F 101/00; B01F 101/25; C12M 1/06

(52) U.S. Cl. CPC B01F 27/073; B01F 27/0722; B01F 27/112; B01F 27/118; B01F 27/191; B01F 27/70; B01F 35/2112; C12M 27/08; B01F 2101/1805; B01F 2101/25

(57) ABSTRACT

Embodiments of the present disclosure relate to a food waste disposer, and more specifically, to a food waste disposer which prevents the formation of a blind spot where food accumulates through the shape of a chamber in which food and microorganisms are accommodated, and improves the efficiency of food decomposition and processing by effectively stirring the food and microorganisms using blades and stirring rods.

### CROSS-REFERENCE TO RELATED PATENT APPLICATION

The present application claims priority under 35 U.S.C. § 119 (a) to Korean patent application number 10-2024-0029493 filed on Feb. 29, 2024, in the Korean Intellectual Property Office, the entire disclosure of which is incorporated by reference herein.

### BACKGROUND OF THE INVENTION

### 1. Field

Embodiments of the present disclosure relate to a food waste disposer, and more specifically, to a food waste disposer which prevents formation of a blind spot where food accumulates through the shape of a chamber in which food and microorganisms are accommodated, and improves the efficiency of food decomposition and processing by effectively stirring the food and microorganisms using blades and stirring rods.

### 2. Description of the Related Art

In general, a certain amount of food waste is discharged daily from each household, restaurant, or the like, and such food waste is either used as livestock feed as it is or disposed of after filtering out the mixed moisture through a simple strainer. This method of processing food waste increases the amount of waste, and if the waste is not disposed of frequently, it produces a foul odor and pollutes the surrounding air.

Recently, efforts to reduce food waste in households and restaurants have been actively promoted, but controlling the gradually increasing amount of waste involves various difficulties, and when it is landfilled, not only is the landfill space limited, but also environmental damage caused by the foul odor and leachate has become extremely serious.

Therefore, a volume-rate waste disposal system has recently been implemented, and it has been regulated that food waste disposers which may induce fermentation or drying of food must be installed in newly constructed group cafeterias or apartment complexes, and each household is also increasingly adopting domestic food waste disposers to reduce food waste.

Food waste with a high moisture content is prone to rotting if left as is, and it emits a foul odor, damaging the surrounding sanitary environment, and many companies have attempted to develop technologies to deal with this, and have released food waste disposer products using various technologies, such as a hot air circulation drying method, a hot air circulation drying method with added stirring function, a hot air circulation drying method with added stirring and grinding function, a decomposing method using a stirring function and microorganisms, and a refrigerated storage method, etc., for processing food waste in households and restaurants.

Among these, a conventional food waste disposer with a general stirring and grinding function includes a processing part in which introduced food waste is processed, and the processing part is provided with a blade that grinds and stirs the food waste.

However, the conventional food waste disposer has a problem in that the blade rotates to stir while moving the food waste to one side, causing a phenomenon of food waste crowding within the processing part, and as a result, the stirring efficiency of the food waste is reduced, and it becomes difficult to discharge the processed food waste.

Further, the conventional food waste disposer has a problem in that, due to the viscosity of the food waste, overall stirring does not occur inside the chamber of the processing part, causing the food waste to stuck to the inner surface or corners, which makes it difficult to discharge the food waste, and only the food waste at the location where the blade is provided is ground and stirred, making it difficult to grind and stir stuck food or food accommodated at a location far from the location of the blade.

In addition, the conventional food waste disposer has a problem in that, since the amount of food waste accommodated inside the processing part is visually checked by a user or an operator when introducing food, even if food is introduced with an input amount exceeding the predetermined capacity, it is difficult to visually identify, and as a result, the stirring and processing efficiency of the food waste disposer decreases, and frequent malfunctions occur to the food waste disposer.

### SUMMARY OF THE INVENTION

The present disclosure has been devised to solve the problems described above, and more specifically, to provide a food waste disposer which prevents formation of a blind spot where food accumulates through the shape of a chamber in which food and microorganisms are accommodated, improves the efficiency of food decomposition and processing by effectively stirring the food and microorganisms using blades and stirring rods, and prevents food from being excessively introduced into the chamber and reducing the decomposition efficiency by providing a distance sensor.

A food waste disposer according to an embodiment of the present disclosure is a food waste disposer for stirring food and microorganisms to process the food to be decomposed, including: a processing part provided with a chamber formed so that a predetermined accommodation space is formed to accommodate food and microorganisms inside and a lower part has a semi-cylindrical shape, a rotating shaft which is rotationally driven inside the chamber, and a stirring rod having one end which is vertically connected to an outer circumferential surface of the rotating shaft and is driven together with the rotating shaft to stir the food and microorganisms accommodated in the chamber; an input part formed on an upper part of the processing part and provided so that the food and microorganisms are introduced from outside into the chamber, in which an input port is formed so that a predetermined area is open; a driving part configured to provide power so that the rotating shaft and the stirring rod are rotationally driven; and a discharge part provided to discharge the food decomposed through the processing part to the outside.

More specifically, the chamber may further include a chamfered part configured to form a sloped surface of a predetermined angle on at least one edge.

More specifically, the rotating shaft may be disposed in a direction perpendicular to a direction in which the food and microorganisms are introduced into the chamber, and have both ends coupled and fixed to center parts of side surfaces of the chamber.

More specifically, the stirring rod may be rotationally driven while moving along a longitudinal direction of the rotating shaft on the outer circumferential surface of the rotating shaft.

More specifically, the rotating shaft may be provided with a screw thread on the outer circumferential surface to form a movement path of the stirring rod.

More specifically, a plurality of the stirring rods may be provided so that each stirring rod is movable in a form of gathering toward a center in the longitudinal direction of the rotating shaft or scattering toward both ends of the rotating shaft.

More specifically, the rotating shaft may further include a plurality of slots formed to be recessed or penetrate to a predetermined depth from the outer circumferential surface toward a center and spaced apart from each other by a predetermined distance in the longitudinal direction of the rotating shaft, and the stirring rod may be provided to be detachably attached to the slot, allowing an installation location to be changed according to a formation location of the slot.

More specifically, the processing part may include a first blade which is coupled to another end of the stirring rod being perpendicular to a longitudinal direction of the stirring rod to form a predetermined contact area to contact the food and microorganisms.

More specifically, the first blade may be disposed at a predetermined angle tilted from the rotating shaft to form an oblique line.

More specifically, the first blade may further include a first brush formed to be detachably attached to one end part of the first blade, on which areas recessed from an outer side toward a center to a predetermined depth are repeatedly formed while being spaced apart from each other by a predetermined distance.

More specifically, the processing part may include a second blade which is coupled to a side surface of the stirring rod being parallel to a longitudinal direction of the stirring rod to form a predetermined contact area to contact the food and microorganisms.

More specifically, the second blade may be disposed so that a width direction is perpendicular to the longitudinal direction of the rotating shaft.

More specifically, the second blade may further include a second brush formed to be detachably attached to one side end part of the second blade, on which areas recessed from an outer side toward a center to a predetermined depth are repeatedly formed while being spaced apart from each other by a predetermined distance.

More specifically, the processing part may include a third blade having a plate shape and provided with a bending part of a predetermined angle, and formed by being coupled to the stirring rod so that both surfaces contact a side surface of the stirring rod based on the bending part to form a predetermined contact area to contact the food and microorganisms.

More specifically, a plurality of the third blades may be provided and disposed to be spaced apart from each other by a predetermined distance in a longitudinal direction of the stirring rod.

More specifically, the input part may include an input cover provided to open and close the input port, and a cover packing formed by being attached to a lower edge of the input cover and provided to seal the chamber when the input port is closed.

More specifically, the cover packing may include at least one protrusion formed to protrude in a vertical direction from one surface toward the outside at a predetermined height and formed along a longitudinal direction of the cover packing.

More specifically, the input part may include a distance sensor mounted in a vertical direction on a lower surface of the input cover and provided to detect a height of the food and microorganisms accommodated inside the chamber when the input port is closed.

More specifically, the distance sensor may be configured to generate a warning notification when the detected height of the food and microorganisms exceeds a preset reference range.

More specifically, the discharge part may include a discharge port provided on one side of the processing part and formed so that a predetermined area is open, and a discharge cover provided to open and close the discharge port.

More specifically, the discharge part may further include a mesh box formed by being coupled to both sides of the discharge port so as to cover the discharge port while forming a predetermined hollow area near the discharge port and provided to be movable up and down based on the discharge port.

More specifically, the discharge cover may be formed so that an upper end is fixedly coupled to an upper part of the discharge port and a lower part is rotatable in an outward direction from the discharge port based on the upper end, and may be provided in a mesh form in which a plurality of holes are formed.

More specifically, the food waste disposer may include a status display part provided to output various information, including information about an operation status of the food waste disposer, information about the food and microorganisms accommodated inside the chamber, and information about a progress status of food processing, in visual and auditory forms.

A food waste disposer according to an embodiment of the present disclosure includes a chamber formed so that the lower part has a semi-cylindrical shape, allowing food and microorganisms inside to gather toward the center while being stirred by stirring rods and blades, thereby preventing the formation of an area in the lower side part where food and microorganisms are not stirred and accumulate, thereby preventing a decrease in the efficiency of decomposition and processing of food.

In particular, a food waste disposer according to an additional embodiment of the present disclosure includes a chamfered part forming an inclined surface at a predetermined angle at the edge of the chamber, thereby preventing the formation of a blind spot or dead zone in which food and microorganisms are not stirred because they do not come into contact with the stirring rod or blade, thereby further improving the efficiency of decomposition and processing of the food waste disposer, and by forming the length of the stirring rod near the chamfered part short, the driving resistance occurring in the stirring rod may be minimized, thereby providing an effect of preventing damage to the food waste disposer.

Further, a food waste disposer according to an embodiment of the present disclosure includes first and second brushes that are formed to be detachably attached to the first and second blades, so that the shape of the blades may be changed when using depending on the type or characteristics of the food to be processed, and food may be prevented from becoming entangled or lumped, so that the stirring of food and microorganisms may be facilitated, thereby providing an effect of improving the speed of food processing.

In addition, the food waste disposer according to an embodiment of the present disclosure includes a protrusion formed along the longitudinal direction of the cover packing, so that when the input cover is closed to close the input port, it adheres more strongly to the inner side surface or peripheral part of the input port, thereby preventing dust generated inside the chamber from leaking out through the input port, thereby providing an effect of further improving the sealing capacity of the space inside the chamber.

In addition, the food waste disposer according to an embodiment of the present disclosure includes a distance sensor that detects the height of food and microorganisms accommodated inside the chamber and generates a warning notification when the height exceeds a reference range, thereby preventing excessive food from being introduced into the chamber and reducing the efficiency of decomposition and processing of the food, and the process of a user directly opening the input cover and visually checking the input amount when using the food waste disposer is unnecessary, thereby providing an effect of increasing user convenience.

In addition, a food waste disposer according to an additional embodiment of the present disclosure includes a mesh box that forms a hollow area near the discharge port and is movable up and down based on the discharge port, thereby ensuring a safe distance for the user or operator from the discharge port, thereby providing an effect of preventing an accident from occurring in advance due to the user's or operator's hand being caught inside the discharge port while the food waste disposer is in operation.

### BRIEF DESCRIPTION OF THE DRAWINGS

FIG. 1 is a perspective view illustrating a food waste disposer according to an embodiment of the present disclosure.

FIG. 2 is a plan view illustrating a state in which an input part of a food waste disposer is separated according to an embodiment of the present disclosure.

FIG. 3 is a front view and a perspective view illustrating a processing part and a driving part of a food waste disposer according to an embodiment of the present disclosure.

FIG. 4 is a plan view illustrating a processing part of a food waste disposer according to an additional embodiment of the present disclosure.

FIG. 5 is a diagram illustrating a detailed configuration of a processing part of a food waste disposer according to an additional embodiment of the present disclosure.

FIG. 6 is a diagram illustrating a detailed configuration of a processing part of a food waste disposer according to another additional embodiment of the present disclosure.

FIG. 7 is a diagram illustrating a first blade and a second blade according to an additional embodiment of the present disclosure.

FIG. 8 is a diagram illustrating a detailed configuration of a processing part of a food waste disposer according to another embodiment of the present disclosure.

FIG. 9 is a diagram illustrating a detailed configuration of an input part of a food waste disposer according to an embodiment of the present disclosure.

FIG. 10 is a cross-sectional view illustrating a cover packing of a food waste disposer according to an embodiment of the present disclosure.

FIG. 11 is a front view illustrating a discharge part of a food waste disposer according to an embodiment of the present disclosure.

FIG. 12 is a perspective view illustrating a discharge part of a food waste disposer according to an additional embodiment of the present disclosure.

FIG. 13 is a diagram illustrating a sequence for opening a discharge part of a food waste disposer according to an additional embodiment of the present disclosure.

FIG. 14 is a perspective view illustrating a discharge part of a food waste disposer according to another embodiment of the present disclosure.

FIG. 15 is a side view illustrating a closed state and an open state of a discharge cover of a discharge part of a food waste disposer according to another embodiment of the present disclosure.

### DETAILED DESCRIPTION

The following detailed description of the present disclosure refers to the accompanying drawings, which illustrate specific embodiments in which the present disclosure may be practiced. These embodiments are described in sufficient detail to enable those skilled in the art to practice the present disclosure. It should be understood that the various embodiments of the present disclosure, while different from one another, are not necessarily mutually exclusive. For example, the specific shapes, structures, and characteristics described herein may be implemented in other embodiments without departing from the spirit and scope of the present disclosure with respect to an embodiment.

In addition, it should be understood that the location or arrangement of individual components within each disclosed embodiment may be changed without departing from the spirit and scope of the present disclosure. Accordingly, the following detailed description is not intended to be limited, and the scope of the present disclosure is defined only by the appended claims, along with the full range of equivalents to which such claims are entitled, if appropriately described. Like reference numerals in the drawings designate the same or similar functions throughout the several aspects.

Hereinafter, in order to enable those skilled in the art to easily practice the present disclosure, preferred embodiments of the present disclosure will be described in detail with reference to the accompanying drawings.

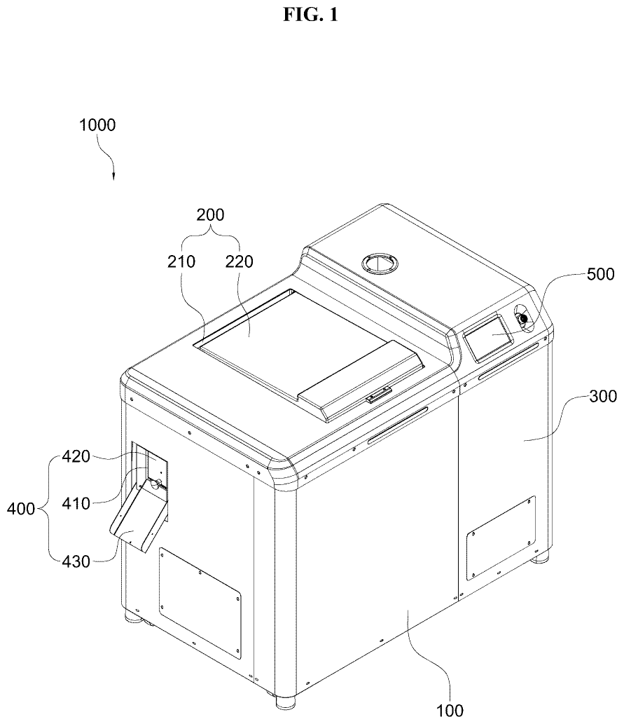

First, a food waste disposer according to an embodiment of the present disclosure will be described with reference to FIGS. 1 to 3.

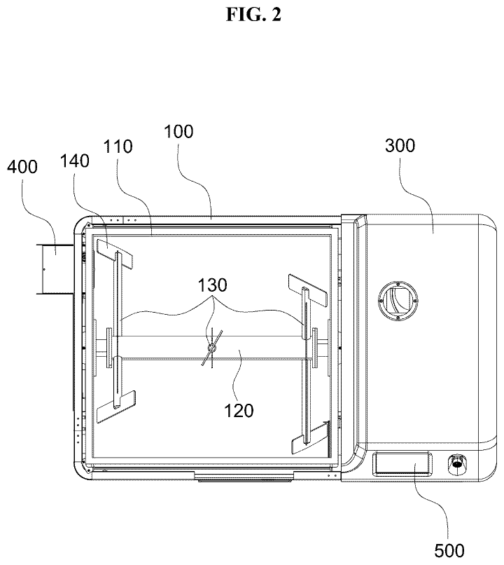

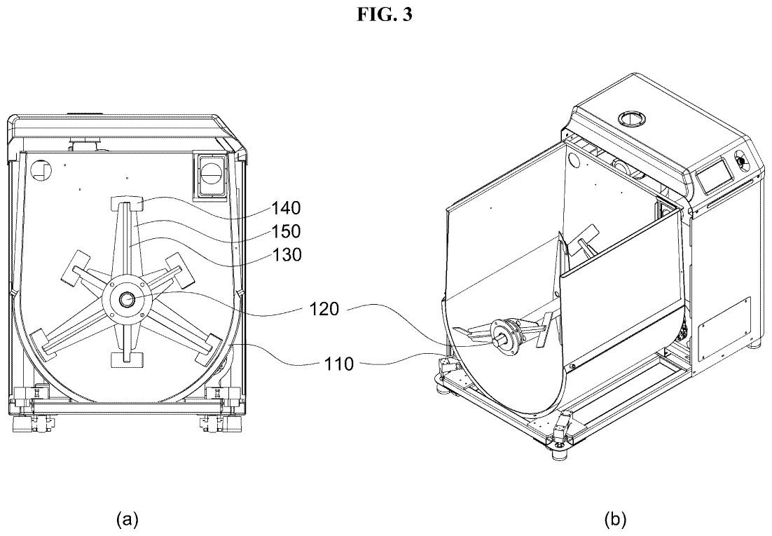

FIG. 1 is a perspective view illustrating a food waste disposer according to an embodiment of the present disclosure, FIG. 2 is a plan view illustrating a state in which an input part of a food waste disposer is separated according to an embodiment of the present disclosure, and FIG. 3 is a front view and a perspective view illustrating a processing part and a driving part of a food waste disposer according to an embodiment of the present disclosure.

The food waste disposer of the present disclosure corresponds to a mechanism and device for stirring food and microorganisms to decompose food.

As illustrated in FIG. 1, a food waste disposer 1000 according to an embodiment of the present disclosure includes a processing part 100, an input part 200, a driving part 300, a discharge part 400, and a status display part 500.

First, the processing part 100 is provided to process food and microorganisms by accommodating and stirring them.

More specifically, the processing part 100 is configured to include a chamber 110, a rotating shaft 120, a stirring rod 130, a first blade 140, and a second blade 150, as illustrated in FIGS. 2 and 3.

Here, the chamber 110 forms a predetermined accommodation space so that food and microorganisms are accommodated inside, and is formed so that the lower part has a semi-cylindrical shape, as illustrated in FIGS. 3a and 3b.

Accordingly, the food waste disposer of the present disclosure includes a chamber 110 formed so that the lower part has a semi-cylindrical shape, thereby allowing the food and microorganisms inside to gather toward the center while being stirred by the stirring rod and the blade, thereby preventing the formation of an area in which the food and microorganisms are not stirred and accumulate in the lower side part, thereby providing an effect of preventing the decomposition and processing efficiency of the food from being reduced.

In addition, the rotating shaft 120 is characterized by being rotationally driven inside the chamber 110.

Here, the rotating shaft 120 is disposed in a direction perpendicular to the direction in which food and microorganisms are introduced into the chamber 110, and both ends are coupled and fixed to center parts of the side surfaces of the chamber 110.

In addition, the stirring rod 130, as illustrated in FIGS. 2 and 3, is provided so that one end is vertically coupled to the outer circumferential surface of the rotating shaft 120 and is driven together with the rotating shaft 120 to stir food and microorganisms accommodated in the chamber 110.

Next, the first blade 140 is characterized by being coupled to the other end of the stirring rod 130 in a direction perpendicular to the longitudinal direction of the stirring rod 130 to form a predetermined contact area to come into contact with food and microorganisms.

More specifically, the first blade 140, as illustrated in FIG. 3, is disposed at a predetermined angle tilted from the rotating shaft 120 to form an oblique line.

In addition, the second blade 150 is characterized by being coupled to a side surface of the stirring rod 130 in a direction parallel to the longitudinal direction of the stirring rod 130 to form a predetermined contact area to come into contact with food and microorganisms.

More specifically, the second blade 150, as illustrated in FIG. 3, is disposed so that the width direction is perpendicular to the longitudinal direction of the rotating shaft 120.

Here, the first blade 140 and the second blade 150 are characterized by being coupled to intersect with each other at a predetermined angle on the stirring rod 130.

As described above, the food waste disposer of the present disclosure may perform the function of separating clumped portions by rotating to separate food and microorganisms inside the chamber in diagonal and vertical directions from top to bottom or bottom to top by the coupled structure of the first blade 140 and the second blade 150, and the food and microorganisms that come into contact with each of the first blade 140 and the second blade 150 may be pushed in different directions and stirred, thereby pushing the food and microorganisms in multi-directions rather than one direction and mixing them, thereby improving the stirring efficiency and the food processing efficiency.

Next, the input part 200 is formed on the upper part of the processing part 100 and is provided so that food and microorganisms are introduced from the outside into the chamber 110.

In addition, the driving part 300 is characterized by providing power so that the rotating shaft 120 and the stirring rod 130 are rotationally driven.

In addition, the discharge part 400 is provided to discharge the food decomposed through the processing part 100 to the outside.

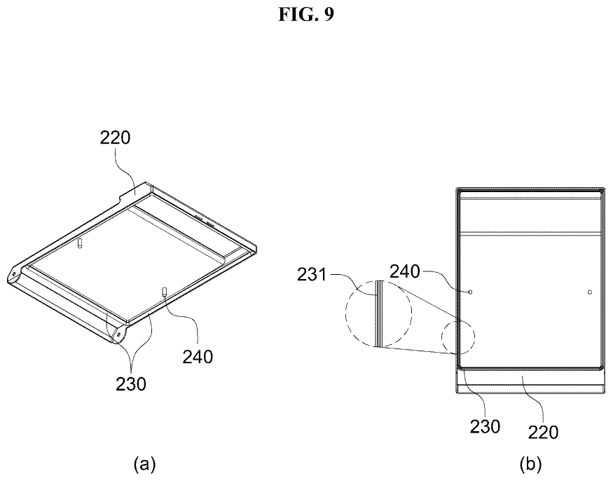

Hereinafter, the input part 200 and the discharge part 400 will be described in more detail with reference to FIGS. 9 to 13 to be described later.

Next, the status display part 500 is characterized by being provided to output various information, including information about an operation status of the food waste disposer 1000, information about the food and microorganisms accommodated inside the chamber 110, and information about a progress status of food processing, in visual and auditory forms.

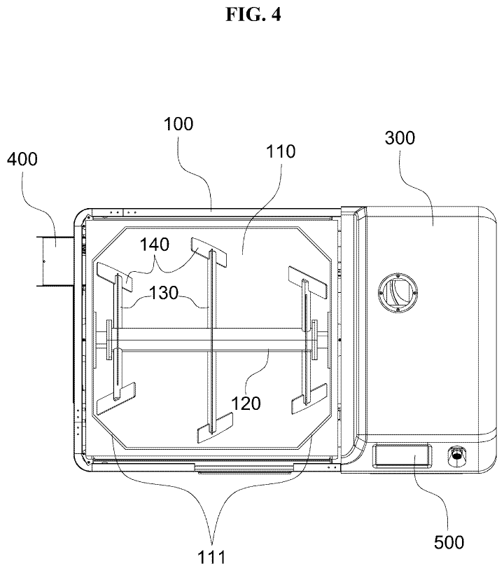

Next, the processing part 100 of the food waste disposer according to an additional embodiment of the present disclosure will be described with reference to FIGS. 4 to 8.

In proceeding with the description of the additional embodiment of the present disclosure, expressions that are redundant with the above-described embodiment of the present disclosure will be omitted for the sake of concise description of the disclosure.

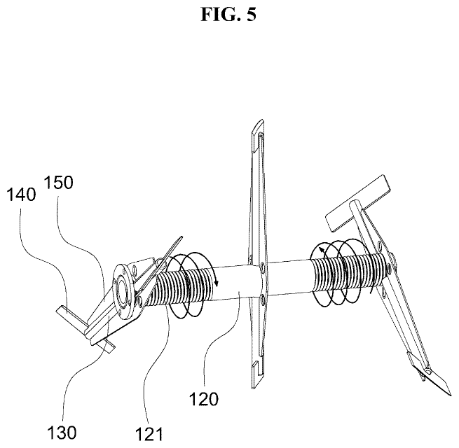

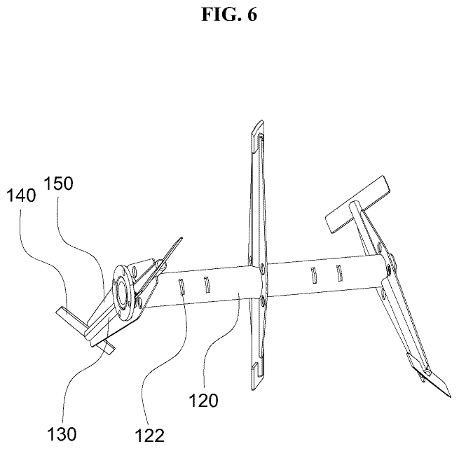

FIG. 4 is a plan view illustrating a processing part of a food waste disposer according to an additional embodiment of the present disclosure, FIG. 5 is a diagram illustrating a detailed configuration of a processing part of a food waste disposer according to an additional embodiment of the present disclosure, and FIG. 6 is a diagram illustrating a detailed configuration of a processing part of a food waste disposer according to another additional embodiment of the present disclosure.

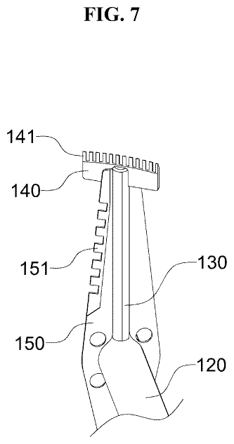

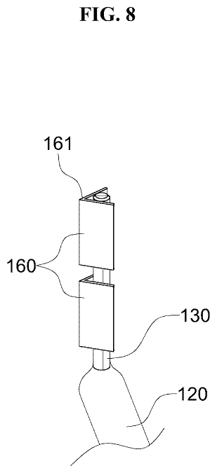

In addition, FIG. 7 is a diagram illustrating a first blade and a second blade according to an additional embodiment of the present disclosure, and FIG. 8 is a diagram illustrating a detailed configuration of a processing part of a food waste disposer according to another embodiment of the present disclosure.

First, as illustrated in FIG. 4, the chamber 110 of the food waste disposer according to an additional embodiment of the present disclosure includes a chamfered part 111.

Here, the chamfered part 111 is provided to form a sloped surface of a predetermined angle on at least one edge of the chamber 110.

At this time, the length of the stirring rod coupled to the vicinity of the area where the chamfered part 111 is formed, i.e., the both end parts of the rotating shaft 120, is formed shorter than the length of the stirring rod coupled to the center part of the rotating shaft 120, so that the driving resistance occurring in the stirring rod at both end parts of the rotating shaft 120 may be minimized.

Accordingly, the food waste disposer of the present disclosure includes the chamfered part 111 formed at the edge of the chamber, thereby preventing the formation of a blind spot or dead zone in which food and microorganisms are not stirred because they do not come into contact with the stirring rod or blade, thereby further improving the efficiency of decomposition and processing of the food waste disposer, and by forming the length of the stirring rod near the chamfered part short, the driving resistance occurring in the stirring rod may be minimized, thereby providing an effect of preventing damage to the food waste disposer.

Next, the rotating shaft 120 of the food waste disposer according to an additional embodiment of the present disclosure is characterized by having a screw thread 121, as illustrated in FIG. 5.

Here, the screw thread 121 is provided on the outer circumferential surface of the rotating shaft 120 to form a movement path of the stirring rod 130.

At this time, the stirring rod 130 is characterized by being rotationally driven while moving along the longitudinal direction of the rotating shaft 120, i.e., along the screw thread 121 described above, on the outer circumferential surface of the rotating shaft 120.

More specifically, a plurality of the stirring rods 130 are provided so that each stirring rod 130 is configured to be movable in a form of gathering toward a longitudinal center of the rotating shaft 120 or scattering toward both ends of the rotating shaft 120.

As described above, in the food waste disposer of the present disclosure, the stirring rod may move in both directions on the outer circumferential surface of the rotating shaft through the screw thread 121, and in order to improve the limitation that stirring is only performed within the rotation radius of the location where the fixed stirring rod is located, the stirring area is expanded by moving the location of the stirring rod, thereby providing the effect of preventing the formation of a blind spot where food and microorganisms accumulate between the stirring rods.

In addition, the rotating shaft 120 of the food waste disposer according to another additional embodiment of the present disclosure is characterized by including a slot 122, as illustrated in FIG. 6.

Here, the slot 122 is formed by recessing or penetrating a predetermined depth from the outer circumferential surface of the rotating shaft 120 toward the center, i.e., the center of a circle based on the longitudinal vertical cross-section of the rotating shaft.

More specifically, a plurality of the slots 122 are provided and characterized by being spaced apart from each other by a predetermined distance in the longitudinal direction of the rotating shaft 120.

Here, the above-described stirring rod 130 is configured to be detachably attached to the slot 122, allowing an installation location to be changed according to a formation location of the slot 122.

For example, the stirring rod 130 may be released from its existing location and moved to a location of the slot 122 close to the longitudinal center of the rotating shaft 120 and coupled using a separate fastening member such as a bolt.

Accordingly, by including the slot 122, the food waste disposer of the present disclosure may improve the decomposition power of food by moving the installation location of the stirring rod according to the type, characteristics, and condition of the food and adjusting the gap between the plurality of stirring rods.

Next, the first blade 140 of the food waste disposer according to an additional embodiment of the present disclosure includes a first brush 141, as illustrated in FIG. 7.

Here, the first brush 141 is formed to be detachably attached to one end part of the first blade 140, on which areas recessed from the outer side toward the center to a predetermined depth are repeatedly formed while being spaced apart from each other by a predetermined distance.

In addition, the second blade 150 of the food waste disposer according to an additional embodiment of the present disclosure includes a second brush 151, as illustrated in FIG. 7.

Here, the second brush 151 is characterized by being formed to be detachably attached to one side end part of the second blade 150, on which areas recessed from the outer side toward the center to a predetermined depth are repeatedly formed while being spaced apart from each other by a predetermined distance.

More specifically, the first brush 141 and the second brush 151 may be made of a hard material such as plastic, or a soft material such as rubber and silicone, and may be applied to various types of food, respectively, or may be used for the purpose of cleaning the inside of the chamber.

As described above, the food waste disposer of the present disclosure includes first and second brushes 141, 151 that are formed to be detachably attached to the first and second blades 140, 150, so that the shape of the blades may be changed when using depending on the type or characteristics of the food to be processed, and food may be prevented from becoming entangled or lumped, so that the stirring of food and microorganisms may be facilitated, thereby providing an effect of improving the speed of food processing.

In addition, the processing part 100 of the food waste disposer according to another embodiment of the present disclosure includes a third blade 160, as illustrated in FIG. 8. Here, the third blade 160 is characterized by having a plate shape and a bending part 161 having a predetermined angle.

In addition, the third blade 160 is formed by being coupled to the stirring rod 130 so that both side surfaces contact the side surface of the stirring rod 130 based on the bending part 161 to form a predetermined contact area to contact the food and microorganisms according to the rotational drive of the rotating shaft 120.

More specifically, as illustrated in FIG. 8, a plurality of the third blade 160 are provided and characterized by being disposed to be spaced apart from each other by a predetermined distance in the longitudinal direction of the stirring rod 130.

Accordingly, by including the third blade 160, the food waste disposer of the present disclosure may evenly flow and stir food and microorganisms to both sides based on the bending part according to the rotation of the stirring rod fixedly coupled to the rotating shaft, and it is applied partially in multiple parts rather than as a single whole in the longitudinal direction of the stirring rod to prevent stress from being concentrated on the stirring rod when rotating, thereby preventing damage to the stirring rod and blade in advance.

Next, an input part of a food waste disposer according to an embodiment of the present disclosure will be described with reference to FIGS. 1, 2, 9, and 10.

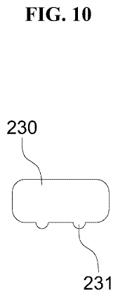

FIG. 9 is a diagram illustrating a detailed configuration of an input part of a food waste disposer according to an embodiment of the present disclosure, and FIG. 10 is a cross-sectional view illustrating a cover packing of a food waste disposer according to an embodiment of the present disclosure.

First, the input part 200, as described above, is formed on the upper part of the processing part 100 described above and provided so that the food and microorganisms are introduced from the outside into the chamber 110.

In addition, the input part 200 includes an input port 210, an input cover 220, a cover packing 230, and a distance sensor 240, as illustrated in FIGS. 1 and 9.

The input port 210 is formed so that a predetermined area of the input part 200 is open.

In addition, the input cover 220 is provided so as to open and close the input port 210.

In addition, the cover packing 230 is characterized in that it is formed by being attached to the lower edge of the input cover 220 and provided to seal the chamber when the input port 210 is closed, as illustrated in FIGS. 9A and 9B.

Here, the cover packing 230 includes a protrusion 231 as illustrated in FIGS. 9B and 10.

More specifically, the protrusion 231 is formed to protrude in the vertical direction from one surface of the cover packing 230 toward the outside at a predetermined height, and is characterized in that at least one is formed along the longitudinal direction of the cover packing 230.

Here, the cover packing 230 is made of a soft material such as rubber or silicone, and when the input cover 220 is closed to close the input port, it contacts the inner side surface or the periphery of the input port and adheres more strongly due to the shape deformation of the protrusion 231, thereby preventing dust generated inside the chamber from leaking out through the input port to further improve the sealing capacity of the space inside the chamber.

Next, the distance sensor 240 mounted in the vertical direction on the lower surface of the input cover 220 and provided to detect the height of the food and microorganisms accommodated inside the chamber when the input port is closed.

More specifically, the distance sensor 240 is characterized by generating a warning notification when the detected height of the food and microorganisms exceeds a preset reference range.

Here, information about the height of the food and microorganisms detected by the distance sensor 240 and information such as the generated warning notification may be output through the status display part described above.

Accordingly, the food waste disposer of the present disclosure includes a distance sensor 240 that detects the height of food and microorganisms accommodated inside the chamber and generates a warning notification when the height exceeds a reference range, thereby preventing excessive food from being introduced into the chamber and reducing the efficiency of decomposition and processing of the food, and the process of a user directly opening the input cover and visually checking the input amount when using the food waste disposer is unnecessary, thereby providing an effect of increasing user convenience.

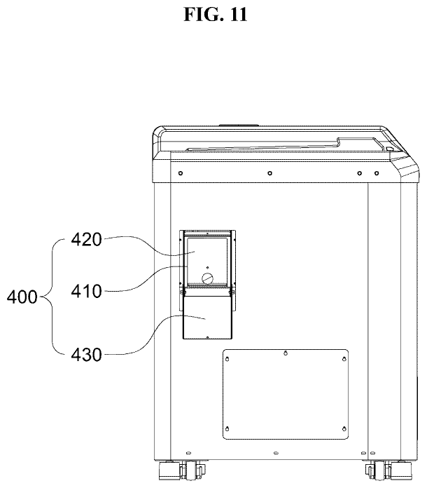

Next, a discharge part of a food waste disposer according to an embodiment, an additional embodiment, and another embodiment of the present disclosure will be described with reference to FIGS. 1 and 11.

FIG. 11 is a front view illustrating a discharge part of a food waste disposer according to an embodiment of the present disclosure.

First, the discharge part 400 is provided to discharge the food decomposed through the above-described processing part 100 to the outside as described above.

In addition, the discharge part 400 includes a discharge port 410, a discharge cover 420, and a stand 430 as illustrated in FIGS. 1 and 11.

Here, the discharge port 410 is provided on one side of the processing part 100 and is formed to so that a predetermined area is open.

In addition, the discharge cover 420 is provided to open and close the discharge port 410.

More specifically, the discharge cover 420 may be formed with a handle that may be gripped by a user or an operator as illustrated in FIGS. 1 and 11.

In addition, the discharge cover 420 is provided so that a part of the upper end is hung on the upper part of the discharge port 410, and when opened, the discharge cover 420 is rotated outward from the discharge port 410 using the handle formed at the lower part so that the discharge cover is perpendicular to the discharge port, and the discharge cover is slid and inserted into the discharge port, thereby completely opening the discharge port.

In addition, the stand 430 is provided so as to form a discharge path while supporting the decomposed food discharged from the discharge port 410.

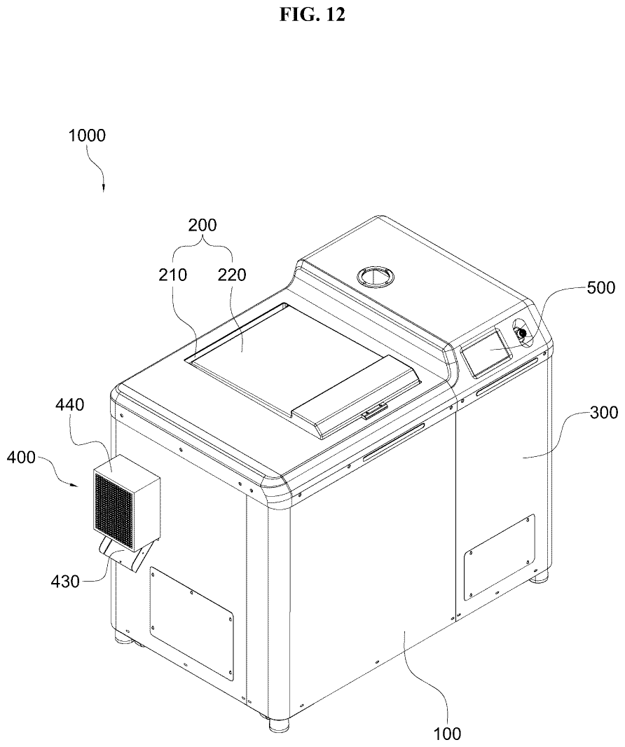

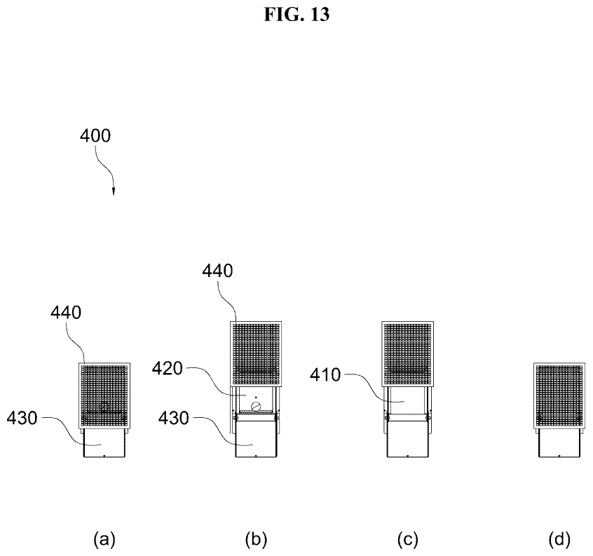

Next, a discharge part of a food waste disposer according to an additional embodiment of the present d disclosure will be described with reference to FIGS. 12 and 13.

FIG. 12 is a perspective view illustrating a discharge part of a food waste disposer according to an additional embodiment of the present disclosure, and FIG. 13 is a diagram illustrating a sequence for opening a discharge part of a food waste disposer according to an additional embodiment of the present disclosure.

First, the discharge part 400 of the food waste disposer of the present disclosure further includes a mesh box 440, as illustrated in FIGS. 12 and 13.

The mesh box 440 is formed by being coupled to both sides of the discharge port so as to cover the discharge port 410 while forming a predetermined hollow area near the discharge port 410.

More specifically, the mesh box 440 is provided to be movable up and down based on the discharge port 410, as illustrated in FIGS. 13B and 13C.

Here, the mesh box 440 is provided in a mesh shape in which a plurality of holes are formed.

Accordingly, the food waste disposer of the present disclosure includes a mesh box 440 that forms a hollow area near the discharge port and is movable up and down based on the discharge port, thereby ensuring a safe distance for the user or operator from the discharge port, thereby providing an effect of preventing an accident from occurring in advance due to the user's or operator's hand being caught inside the discharge port while the food waste disposer is in operation.

The opening of the discharge part 400 using the mesh box 440 is performed in the order of FIGS. 13A to 13D.

FIG. 13A is a diagram illustrating a state in which the discharge port is closed by the mesh box 440 and the discharge cover, and FIG. 13B is a diagram illustrating a state in which the discharge port is closed only by the discharge cover 420 by moving the mesh box 440 up based on the discharge port 410.

Here, the mesh box 440 may be coupled so that only the left and right side parts are hung around the discharge port 410, and moved by sliding when moving up and down.

Next, FIG. 13C illustrates a state in which the discharge port 410 is completely opened by opening the discharge cover 420 while the mesh box 440 is moved up, and FIG. 13D illustrates a state in which the mesh box 440 is moved down again to form a predetermined hollow area near the discharge port 410 and cover the discharge port 410.

Afterwards, when the decomposed food is discharged from the inside of the chamber of the above-described processing part to the discharge port, the front surface of the discharge port 410 is covered by the mesh box 440, and the food flows out to the lower part of the discharge port, i.e., the stand 430, so that safe discharge of the food is possible.

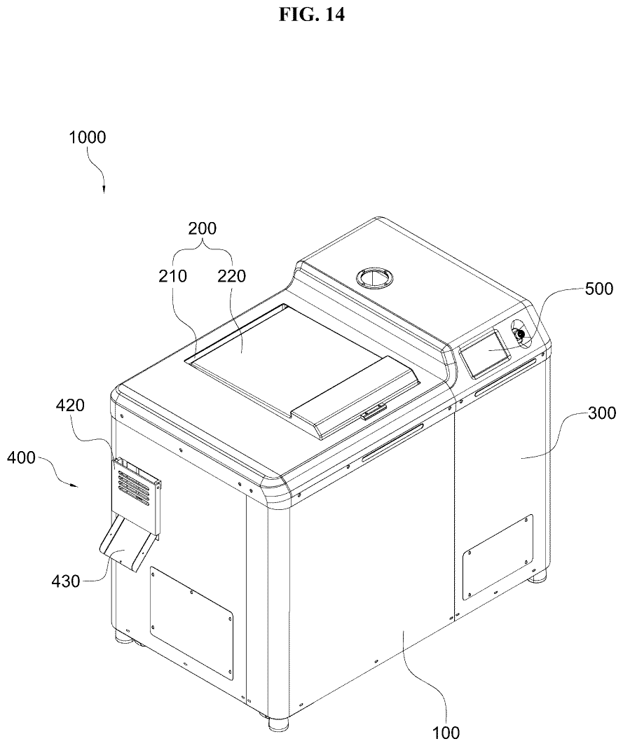

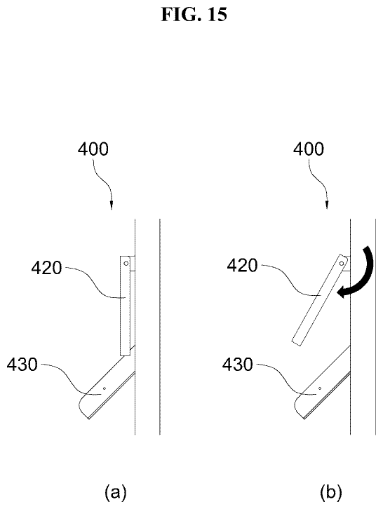

Next, a discharge part of a food waste disposer according to another embodiment of the present disclosure will be described with reference to FIGS. 14 and 15.

FIG. 14 is a perspective view illustrating a discharge part of a food waste disposer according to another embodiment of the present disclosure, and FIG. 15 is a side view illustrating a closed state and an open state of a discharge cover of a discharge part of a food waste disposer according to another embodiment of the present disclosure.

First, the discharge cover 420 of the food waste disposer of the present disclosure may be provided in a mesh shape in which a plurality of holes are formed, as illustrated in FIGS. 14 and 15.

More specifically, the discharge cover 420 is characterized by, as illustrated in FIG. 15, being formed so that the upper end is fixedly coupled to the upper part of the discharge port and the lower part is rotatable in the outward direction from the discharge port based on the upper end.

Here, FIG. 15A is a diagram illustrating the discharge part 400 in a state where the discharge port is closed by the discharge cover 420, and FIG. 15B is a diagram illustrating the discharge part 400 in a state where the discharge port is opened by the discharge cover 420.

More specifically, the opening of the discharge port is performed in a form in which, as illustrated in FIG. 15B, the discharge cover 420 is fixed to the upper end, the decomposed food is pushed out from inside the chamber to the discharge port, and the lower part of the discharge cover 420 rotates due to the weight of the food, thereby opening the discharge port.

Accordingly, the food waste disposer of the present disclosure includes a discharge cover 420 provided in a mesh form, thereby ensuring a safe distance for the user or operator from the discharge port, and providing an effect of preventing a safety accident from occurring when the user or operator's hand gets caught inside the discharge port during the operation and driving of the food waste disposer.

Although the present disclosure has been described above with specific details such as specific components, limited embodiments, and drawings, these have been provided only to help a more general understanding of the present disclosure, and the present disclosure is not limited to the above embodiments, and those skilled in the art may attempt various modifications and variations from this description.

Therefore, the idea of the present disclosure should not be limited to the embodiments described above, and not only the appended claims but also all modifications equivalent to or equivalent to the claims are included in the scope of the idea of the present disclosure.

<Explanation of Symbols>

| | |
|---|---|
| 1000: Food waste disposer | |
| 100: Processing part | 110: Chamber |
| 111: Chamfered part | 120: Rotating shaft |
| 121: Screw thread | 122: Slot |
| 130: Stirring rod | 140: First blade |
| 141: First brush | 150: Second blade |
| 151: Second brush | 160: Third blade |
| 161: Bending part | 200: Input part |
| 210: Input port | 220: Input cover |
| 230: Cover packing | 231: Protrusion |
| 240: Distance sensor | 300: Driving part |
| 400: Discharge part | 410: Discharge port |
| 420: Discharge cover | 430: Stand |
| 440: Mesh box | 500: Status display part |

What is claimed is:

1. A food waste disposer for stirring food and microorganisms to process the food to be decomposed, the food waste disposer comprising:
a processing part 100 provided with a chamber 110 formed so that a predetermined accommodation space is formed to accommodate food and microorganisms inside and a lower part has a semi-cylindrical shape, a rotating shaft 120 which is rotationally driven inside the chamber, and a stirring rod 130 having one end which is vertically connected to an outer circumferential surface of the rotating shaft and is driven together with the rotating shaft to stir the food and microorganisms accommodated in the chamber;
an input part 200 formed on an upper part of the processing part and provided so that the food and microorganisms are introduced from outside into the chamber, in which an input port 210 is formed so that a predetermined area is open;
a driving part 300 configured to provide power so that the rotating shaft and the stirring rod are rotationally driven; and
a discharge part 400 provided to discharge the food decomposed through the processing part to the outside,
wherein the chamber 110 is provided with a chamfered part 111 configured to form a sloped surface of a predetermined angle on at least one edge,
the stirring rod 130 is rotationally driven while moving along a longitudinal direction of the rotating shaft on the outer circumferential surface of the rotating shaft, and a plurality of the stirring rods are provided so that each stirring rod is movable in a form of gathering toward a center in the longitudinal direction of the rotating shaft or scattering toward both ends of the rotating shaft, and
the rotating shaft 120 is provided with a screw thread 121 on the outer circumferential surface to form a movement path of the stirring rod.

2. The food waste disposer according to claim 1, wherein the rotating shaft 120 is disposed in a direction perpendicular to a direction in which the food and microorganisms are introduced into the chamber and has both ends coupled and fixed to center parts of side surfaces of the chamber.

3. The food waste disposer according to claim 1, wherein the rotating shaft 120 further comprises a plurality of slots 122 formed to be recessed or penetrate to a predetermined depth from the outer circumferential surface toward a center and spaced apart from each other by a predetermined distance in the longitudinal direction of the rotating shaft, and
the stirring rod 130 is provided to be detachably attached to the slot, allowing an installation location to be changed according to a formation location of the slot.

4. The food waste disposer according to claim 1, wherein the processing part 100 comprises a first blade 140 which is coupled to another end of the stirring rod being perpendicular to a longitudinal direction of the stirring rod to form a predetermined contact area to contact the food and microorganisms.

5. The food waste disposer according to claim 4, wherein the first blade 140 is disposed at a predetermined angle tilted from the rotating shaft to form an oblique line.

6. The food waste disposer according to claim 4, wherein the first blade 140 further comprises a first brush 141 formed to be detachably attached to one end part of the first blade, on which areas recessed from an outer side toward a center to a predetermined depth are repeatedly formed while being spaced apart from each other by a predetermined distance.

7. The food waste disposer according to claim 1, wherein the processing part 100 comprises a second blade 150 which is coupled to a side surface of the stirring rod being parallel to a longitudinal direction of the stirring rod to form a predetermined contact area to contact the food and microorganisms.

8. The food waste disposer according to claim 7, wherein the second blade 150 is disposed so that a width direction is perpendicular to the longitudinal direction of the rotating shaft.

9. The food waste disposer according to claim 7, wherein the second blade 150 further comprises a second brush 151 formed to be detachably attached to one side end part of the second blade, on which areas recessed from an outer side toward a center to a predetermined depth are repeatedly formed while being spaced apart from each other by a predetermined distance.

10. The food waste disposer according to claim 1, wherein the processing part 100 comprises a third blade 160 having a plate shape and provided with a bending part 161 of a predetermined angle, and formed by being coupled to the stirring rod so that both surfaces contact a side surface of the stirring rod based on the bending part to form a predetermined contact area to contact the food and microorganisms.

11. The food waste disposer according to claim 10, wherein a plurality of the third blades 160 are provided and disposed to be spaced apart from each other by a predetermined distance in a longitudinal direction of the stirring rod.

12. The food waste disposer according to claim 1, wherein the input part 200 comprises:
an input cover 220 provided to open and close the input port; and
a cover packing 230 formed by being attached to a lower edge of the input cover and provided to seal the chamber when the input port is closed.

13. The food waste disposer according to claim 12, wherein the cover packing 230 comprises at least one protrusion 231 formed to protrude in a vertical direction from one surface toward the outside at a predetermined height and formed along a longitudinal direction of the cover packing.

14. The food waste disposer according to claim 12, wherein the input part 200 comprises a distance sensor 240 mounted in a vertical direction on a lower surface of the input cover and provided to detect a height of the food and microorganisms accommodated inside the chamber when the input port is closed.

15. The food waste disposer according to claim 14, wherein the distance sensor 240 is configured to generate a warning notification when the detected height of the food and microorganisms exceeds a preset reference range.

16. The food waste disposer according to claim 1, wherein the discharge part 400 comprises:
a discharge port 410 provided on one side of the processing part and formed so that a predetermined area is open; and
a discharge cover 420 provided to open and close the discharge port.

17. The food waste disposer according to claim 16, wherein the discharge part 400 further comprises a mesh box 440 formed by being coupled to both sides of the discharge port so as to cover the discharge port while forming a predetermined hollow area near the discharge port and provided to be movable up and down based on the discharge port.

18. The food waste disposer according to claim 16, wherein the discharge cover 420 is formed so that an upper end is fixedly coupled to an upper part of the discharge port and a lower part is rotatable in an outward direction from the discharge port based on the upper end, and is provided in a mesh form in which a plurality of holes are formed.

19. The food waste disposer according to claim 1, further comprising a status display part 500 provided to output various information, including information about an operation status of the food waste disposer, information about the food and microorganisms accommodated inside the chamber, and information about a progress status of food processing, in visual and auditory forms.
